(19) United States
(12) Patent Application Publication
(10) Pub. No.: US 2010/0256074 A1
(43) Pub. Date: Oct. 7, 2010

(54) Processes for preparing high purity aza cyclohexapeptides

(76) Inventors: Chaim Eidelman, Oshrat (IL); Avi Tovi, Zurit (IL); Hagai Alon, Kibbutz Yehiam (IL); Vitali Pirogov, Arad (IL); Adelina Gelfond, Keryat Yam (IL); Csilla Nemethne Racz, Tiszavasvari (HU); Ferenc Korodi, Debrecen (HU); Piroska Kovacs, Debrecen (HU); Amir Gold, Herzelia (IL); Alexander Rudinski, Beer-Sheva (IL)

Correspondence Address: KENYON & KENYON LLP, ONE BROADWAY, NEW YORK, NY, 10004, US

(21) Appl. No.: 12/459,180

(22) Filed: Jun. 25, 2009

Related U.S. Application Data
(60) Provisional application No. 61/133,184, filed on Jun. 25, 2008.
(60) Provisional application No. 61/133,602, filed on Jun. 30, 2008.
(60) Provisional application No. 61/174,289, filed on Apr. 30, 2009.

(51) Int. Cl. A61K 38/12; C07K 7/56; A61P 31/10

(52) U.S. Cl. 514/21.1; 530/317

(57) ABSTRACT

Provided are processes for the preparation and purification of high purity aza cyclohexapeptides.

### CROSS REFERENCE

The present invention claims the benefit of the following United States Provisional Patent Application Nos.: 61/133,184 filed Jun. 25, 2008; 61/133,602 filed Jun. 30, 2008; and 61/174,289 filed Apr. 30, 2009. The contents of these applications are incorporated herein by reference.

### FIELD OF THE INVENTION

The invention encompasses processes for the preparation and purification of high purity aza cyclohexapeptides.

### BACKGROUND OF THE INVENTION

Aza cyclohexapeptide compounds are macrocyclic lipopeptides belonging to the echinocandin family, which are useful in treating systemic fungal infections, especially those caused by Candida, Aspergillus, Histoplasma, Coccidioides and Blastomyces. They have also been found useful for the treatment and prevention of infections caused by Pneumocystis carinii which are often found in immunocompromised patients such as those with AIDS. Pneumocandins are a subset of echinocandins which are naturally produced by the fungus Glarea lozoyensis. Their isolation, structure elucidation and biological evaluation have been reported by Schmatz et al in Cutaneous Antifungal Agents, 1993, pp 375-394. Pneumocandin B₀ is a secondary metabolite produced by the fungus Glarea lozoyensis, previously identified as Zalerion arboricola (see e.g. U.S. Pat. Nos. 5,194,377 and 5,202,309), and serves as an intermediate in the production of caspofungin. Pneumocandin B₀, 1-[4,5-dihydroxy-N²-(10,12-dimethyl-1-oxotetradecyl)ornithine]-5-(3-hydroxyglutamine)-6-[3-hydroxy prolinejechinocandin B, with its preferred stereoisomer being 1-[4,5-dihydroxy-N²-(10,12-dimethyl-1-oxotetradecyl)-L-ornithine]-5-(3-hydroxy-L-glutamine)-6-[3-hydroxy-L-proline] echinocandin B is described e.g. in U.S. Pat. No. 5,202,309.

Processes for the preparation of aza cyclohexapeptide compounds are described in WO 94/21677, WO 96/24613, U.S. Pat. No. 5,552,521, WO 97/47645, U.S. Pat. No. 5,936,062 and WO 02/083713.

These patents differ from each other by the synthetic preparation scheme but are similar in regards to the workup of the crude product including isolation and purification of the product. The final product according to these patents is obtained either by lyophilization of the aqueous peptide solution to obtain amorphous powder or by its crystallization as a pharmaceutically acceptable salt.

Suitable pharmaceutically acceptable salts as acid addition salts include acids such as hydrochloric, hydrobromic, phosphoric, sulfuric, maleic, citric, acetic, tartaric, succinic, oxalic, malic, glutamic and the like, and other acids related to the pharmaceutically acceptable salts listed in Journal of Pharmaceutical Science, 66, 2 (1977).

Caspofungin diacetate, 1-[(4R,5S)-5-[(2-aminoethyl)amino]-N2-(10,12-dimethyl-1-oxotetradecyl)-4-hydroxy-L-ornithine]-5-[(3R)-3-hydroxy-L-ornithine]-pneumocandin Bo, is described according to the provided below formula I.:

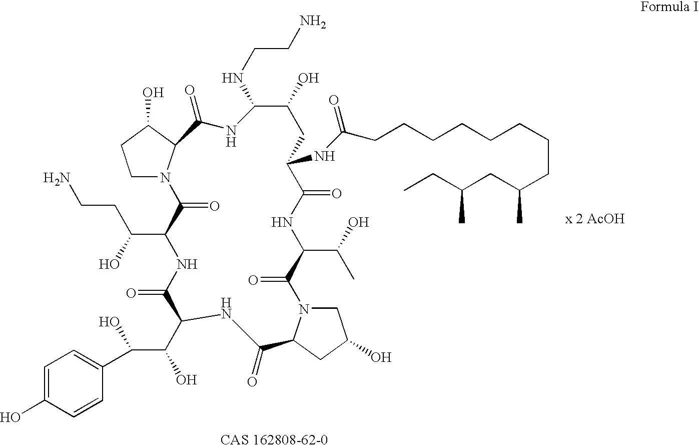

Caspofungin diacetate is a known antifungal and anti-protozoal agent and is marketed by Merck & Co. Inc. Caspofungin is approved for the prevention and/or treatment of infections caused by filamentous fungi and yeasts such as Aspergillus sp., Histoplasma sp., Coccidioides sp., Blastomyces sp. and/or Candida sp., as well as in preventing and/or controlling and/or treating infections such as pneumonia caused by Pneumocystis jiroveci (previously classified as Pneumocystis carinii). Caspofungin may be administered parenterally, e.g. intravenously by use of compositions being either lyophilized or liquid formulations. Caspofungin can be found in a variety of pharmaceutically acceptable salts such as caspofungin diacetate as described e.g. in European Patent EP 0 904 098 B1. Caspofungin and methods for its preparation are described e.g. in WO 94/21677 and EP 620232 which disclose pharmaceutically acceptable salts of aza cyclohexapeptide including caspofungin, as being particularly useful in the control of mycotic infections. WO96/24613 discloses additional processes to prepare caspofungin and in particular caspofungin diacetate salt.

While the poor stability of these products is mentioned in some of the references above (for example WO 97/39763), no specific attention was drawn to prevent possible degradation of the product during the work-up procedures or to define its final purity as a main target of the preparation process.

Several attempts to improve the stability of the formulations were described in some references (WO 97/39763, WO/2008/012310, EP 0 904 098 B1 or U.S. Pat. No. 5,952,300). However, none of them was targeted towards prevention of the degradation during the preparation process of an active compound or its manufacture as a pure product. WO/2008/012310 describes preparation of dipropionate salt of caspofungin in a lyophilized or crystalline form. The dipropionate salt is obtained by dissolution of diacetate salt of caspofungin in solution of organic solvent and water, exchange to propionate salt, and lyophilization to obtain dry product. It also describes further treatment to obtain crystalline form of the caspofungin dipropionate salt.

There is a need in the art for improved processes for the preparation of aza cyclohexapeptides in a more industrially applicable production in terms of purity, stability, amount of degradation products and also costs of production.

### SUMMARY OF THE INVENTION

In one embodiment, the present invention encompasses a process for preparing aza cyclohexapeptide salts comprising spray drying a solution of a pharmaceutically acceptable salt of aza cyclohexapeptide in an organic solvent.

In another embodiment, the present invention provides a process for preparing aza cyclohexapeptide salts comprising:
(a) providing a solution of a pharmaceutically acceptable salt of aza cyclohexapeptide in an organic solvent;
(b) removing an excess of water by evaporating a portion of the solution;
(c) adding anhydrous organic solvent; and
(d) repeating steps (b) and (c) one or more times.

As used herein the term “anhydrous organic solvent” refers to organic solvent having less than 2% water by volume.

Preferably the aza cyclohexapeptide salt is caspofungin diacetate.

Preferably, the obtained caspofungin diacetate from the processes above is of high purity, having at least about 99.0% purity as determined by area by HPLC, preferably at least about 99.5% area by HPLC and less than 0.25% area by HPLC of each of the degradation or process impurities, preferably less than 0.25% area by HPLC, and more preferably less than 0.1% area by HPLC.

In another embodiment, the present invention encompasses a crystalline form of caspofungin diacetate, characterized by data selected from the group consisting of: a powder XRD pattern with peaks at about 3.1, 5.2, 6.1 and 9.0±0.2 degrees two-theta, a powder XRD pattern substantially as depicted in FIG. 1; and combinations thereof.

In one embodiment, the invention encompasses a pharmaceutical composition comprising the above-described crystalline caspofungin diacetate polymorph and at least one pharmaceutically acceptable excipient.

In another embodiment, the invention encompasses a process for preparing a pharmaceutical composition comprising the above-described crystalline caspofungin diacetate polymorph comprising combining the above-described crystalline caspofungin diacetate polymorph and at least one pharmaceutically acceptable excipient.

In yet another embodiment, the invention encompasses a method of treating systemic fungal infections comprising administering to a patient in need thereof a pharmaceutical composition comprising the above-described crystalline caspofungin diacetate polymorph and at least one pharmaceutically acceptable excipient.

### BRIEF DESCRIPTION OF THE FIGURES

FIG. 1. depicts characteristic X-ray powder diffraction pattern of crystalline caspofungin diacetate polymorph.

FIG. 2. depicts characteristic TGA thermogram of the above-described crystalline caspofungin diacetate polymorph.

FIG. 3. depicts characteristic DSC thermogram of the above-described crystalline caspofungin diacetate polymorph.

### DETAILED DESCRIPTION OF THE INVENTION

The invention encompasses processes for preparing aza cyclohexapeptide salts with high purity. More specifically, the invention encompasses processes for the preparation of caspofungin diacetate.

The spray drying technique, although involving working under high temperatures, does not cause degradation of the peptide as would have been expected due to the high sensitivity of the peptide to heat. Instead, it was found that the spray drying affords a dry material having a low level of impurities.

The spray drying technique also serves to remove excess of acetic acid to obtain the material as diacetate (within specified limits).

As used herein, the term “process impurities” refers to protecting groups and excess of reagents.

As used herein, the term “pharmaceutically acceptable counter-ion” or “pharmaceutically acceptable salt” refers to counter-ions or salts such as citrate, acetate, palmitate, trifluoroacetate, hydrochloride, maleate, tartrate, succinate, oxalate, malate or glutamate.

Aza cyclohexapeptides are known in the art, and are disclosed for example in U.S. Pat. Nos. 7,166,572, 5,792,746, 5,668,105, 5,516,756, 5,514,651, 5,514,650, 5,430,018, 5,378,804, 5,310,873, and 5,159,059, all of which are incorporated herein by reference. The aza cyclohexapeptide has the following structure:

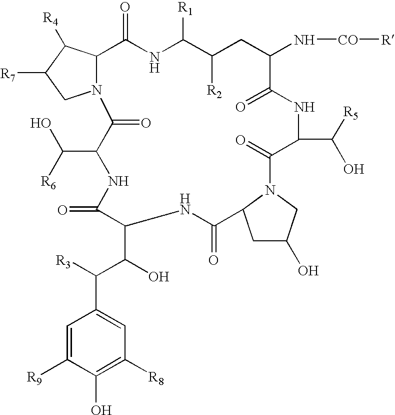

R′ is C₉-C₂₀ branched or straight chain alkyl; C₉-C₂₀ alkenyl; C₉-C₂₀ alkoxyphenyl; an aryl group selected from: phenyl, biphenyl, terphenyl and naphthyl; C₁-C₁₂ alkylphenyl, C₂-C₁₂ alkenylphenyl, C₂-C₁₂ alkoxyterphenyl; C₂-C₁₂ alkoxyphenyloxazolylphenyl; C₁-C₁₂ alkoxyphenyl; linoleoyl; palmitoyl; 12-methylmyristoyl; 10,12-dimethylmyristoyl; or —COC₆H₄(p)OC₈H₁₇;
R₁ and R₃ are independently —H; —OH; —CN; —(CH₂)ₙNH₂, where n is 1-5; —NR₁₀R₁₁ and wherein R₁₀ and R₁₁ are independently H, C₁-C₈ alkyl, C₃-C₄ alkenyl, (CH₂)₂₋₄OH or (CH₂)₂₋₄NR₁₂R₁₃; wherein R₁₂ and R₁₃ are independently H or C₁-C₈ alkyl; or wherein NR₁₀R₁₁ form a heterocyclic ring and R₁₀ and R₁₁ together are (CH₂)₄, (CH₂)₅, (CH₂)₂O(CH₂)₂ or (CH₂)₂NH(CH₂)₂; —N₃; aryl; substituted aryl; heterocyclyl and substituted heterocyclyl with 1-3 of the same or different heteroatoms; aminoalkylamino; mono- or di-substituted linear or cyclic aminoalkylamino; —OR, wherein, R is C₁-C₁₂ alkyl; substituted alkyl of the type —(CH₂)ₙ—X, where n is 1-5 and X is Cl, Br, I, COOY, CN, NH₂ or a heterocyclic and where Y is C₁-C₆ linear or branched alkyl; C₂-C₁₂-alkenyl; aryl; fused aryl; substituted aryl; a heterocyclic containing 1-3 heteroatoms; mono- or di-substituted aminoalkyl; or a hydroxy, protecting group; or R₃ is imidazolyl

### Y is H—, CH₃— or —CH₂ CONH₂;

R₂ and R₄ are independently —H or —OH;

### R₅ is —H or —CH₃;

R₆ is —H, —CH₃, —(CH₂)nNRR″ where n is 1-4 and R and R″ are H, or C₁-C₆ alkyl or —(CH₂)nCONRR″ where n is 1-4 and R and R″ are H, or C₁-C₆ alkyl; R₇ is —H, —CH₃ or —OH; R₈ and R₉ are independently —OH—SO₂—O, Na—SO₃—O—H or —CH₂-secondary amine, the secondary amine being attached to —CH₂ through its N-linkage; and its pharmaceutically acceptable salts.

To the nitrogen atom of the secondary amine are attached the same or different groups selected from: C₁-C₁₂ alkyl, C₂-C₁₂ alkenyl, aryl, substituted aryl, alkylaryl and substituted alkylaryl, or the nitrogen atom of the secondary amine is part of a heterocyclic group, optionally substituted by one or more of: C₁-C₆ alkyl, C₂-C₆ alkenyl, aryl, amino, nitro and halogen, or a fused heterocyclic group, whereby the heterocyclic group in each case contains 1-3 of the same or different heteroatoms.

Caspofungin is shown below:

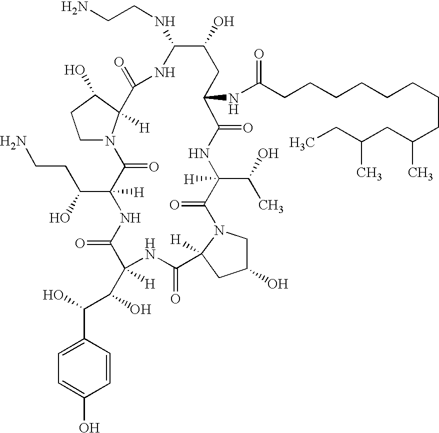

In one embodiment, the present invention encompasses a process for preparing aza cyclohexapeptide salts comprising spray drying a solution of a pharmaceutically acceptable salt of aza cyclohexapeptide in an organic solvent.

Preferably, the solution of a pharmaceutically acceptable salt of aza cyclohexapeptide in an organic solvent may be obtained by combining an aza cyclohexapeptide with a mixture of an organic solvent and an acid of a pharmaceutically acceptable salt or by combining a pharmaceutically acceptable salt of aza cyclohexapeptide with an organic solvent.

Without limiting to any mechanism, those skilled in the art would understand that the acid introduced into the solution is a suitable acid modifier, which potentially forms an addition salt of the aza cyclohexapeptide.

A person skilled in the art would also understand that wherein a solution of an aza cyclohexapeptide salt is spray dried, an acid corresponding to the acid forming the aza cyclohexapeptide salt, is used.

Preferably, the organic solvent is selected from the group consisting of: ethanol, acetonitrile, methanol, propanol, isopropanol, t-butyl alcohol, tetrahydrofuran and mixtures of organic solvent and water. More preferably, the organic solvent is ethanol.

If an aqueous organic solvent is used, the ratio of a the organic solvent and water can be from 50:50 to about 95:5 organic solvent: water, more preferably 80:20 to about 95:5 organic solvent: water.

Most preferably, a mixture of an organic solvent and water at a ratio of about 90:10 to about 95:5 organic solvent: water is used.

The acid used, is in a ratio of about 0.025% to about 0.1% of the total mixture volume. Most preferably 0.05%.

The aza cyclohexapeptides are preferably selected from the list selected from: caspofungin, anidulafungin and micafungin.

Most preferred aza cyclohexapeptide salt, prepared by the above process is caspofungin diacetate.

Preferably, the obtained caspofungin diacetate is amorphous caspofungin diacetate.

Preferably, the obtained caspofungin diacetate is of high purity, having at least about 99.0% by area purity as determined by HPLC, preferably at least about 99.5% area by HPLC and less than 0.3% area by HPLC of each of the degradation or the synthesis process impurities (including CPF-Dimer 1 at RRT 2.08 and CPF-dimer 2 at RRT 2.26), preferably less than 0.25% area by HPLC, and more preferably less than 0.1% area by HPLC.

Preferably, the obtained amorphous caspofungin diacetate contains less than 5% of crystalline caspofungin diacetate described below, more preferably less than 1% of crystalline caspofungin diacetate described below, most preferably, less than 0.5% of crystalline caspofungin diacetate described below, as measured by XRD.

In one specific embodiment, the present invention encompasses a process for preparing caspofungin diacetate comprising spray drying a solution of caspofungin diacetate in ethanol.

Preferably, the solution of caspofungin diacetate in ethanol is obtained by combining caspofungin with a mixture of ethanol and acetic acid or by combining a caspofungin diacetate with ethanol.

Preferably, the solution is spray-dried at an inlet temperature of from about 50° C. to about 200° C., more preferably from about 50° C. to about 150° C. Most preferably, the solution is spray-dried at an inlet temperature of about 80° C.

Preferably, the solution is spray-dried at an outlet temperature of from about 10° C. to about 65° C., more preferably from about 20° C. to about 55° C., and most preferably from about 30° C. to about 45° C.

The obtained product may be collected and kept under cold conditions of less than about −70° C. to further prevent degradation.

The term “spray drying” broadly refers to processes involving breaking up liquid mixtures into small droplets (atomization) and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pgs. 20-54 to 20-57 (Sixth Edition 1984), which is incorporated herein by reference.

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark), and BUCHI Model B-290 mini spray dryer.

As used herein, an “inlet temperature” is the temperature at which the hot gas enters the spray dryer; an “outlet temperature” is the temperature at which the gas exits the spray dryer.

Inlet or outlet temperatures may be varied, if necessary, depending on the equipment, gas, or other experimental parameters. For example, it is known that the outlet temperature may depend on parameters such as aspirator rate, air humidity, inlet temperature, spray air flow, feed rate, or concentration.

Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. The process of the invention is not limited to the use of such drying apparatuses as described above.

Spray-drying may be performed in a conventional manner in the processes of the present invention (see, e.g., Remington: The Science and Practice of Pharmacy, 19th ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The amorphous caspofungin product produced by spray-drying may be recovered by techniques commonly used in the art, such as using a cyclone or a filter.

In another embodiment, the present invention provides a process for preparing aza cyclohexapeptide salts comprising:
(a) providing a solution of a pharmaceutically acceptable salt of aza cyclohexapeptide in an organic solvent;
(b) removing an excess of water by evaporating a portion of the solution;
(c) adding anhydrous organic solvent; and
(d) repeating steps (b) and (c) one or more times.

Preferably, the solution of a pharmaceutically acceptable salt of aza cyclohexapeptide in an organic solvent is obtained by combining an aza cyclohexapeptide with a mixture of an organic solvent and an acid of a pharmaceutically acceptable salt or by combining a pharmaceutically acceptable salt of aza cyclohexapeptide with an organic solvent.

The evaporation cycles are stopped when the final water content is less then 1% wt %. The final water content may be measured using conventional methods such as for example, Karl Fischer (KF).

Preferably in step (b) the solution is evaporated to achieve a volume of about 15%-25% of the starting solution of step (a). Most preferably the solution is evaporated to achieve a volume of about 20% of the starting solution.

The total amount of anhydrous organic solvent added in one or more cycles can be from about 1 volume to about 3 volumes of the starting solution of step (a). Preferably, 2 volumes of anhydrous organic solvent are used.

The process may comprise a further step of drying the peptide from residual solvent by complete evaporation to obtain a powder.

Optionally, moderate heating is also used in the further step of drying. Preferably heating may be performed at a temperature of about 32° C. to about 38° C., most preferably 35° C.

Optionally, the process for the preparation of aza cyclohexapeptide salts by evaporation cycles, as described above, may further comprise the steps of re-dissolution and re-drying by evaporation. These steps may be performed in order to achieve the required water and residual solvent content.

Preferably, the organic solvent used in this process is as described above. An organic solvent which is suitable for the process is a solvent that can dissolve the product and its evaporation results in partial removal of water.

Preferably, the aza cyclohexapeptide salt is caspofungin diacetate.

Preferably, the obtained caspofungin diacetate is amorphous caspofungin diacetate.

Preferably, the obtained amorphous caspofungin diacetate is of high purity as described above.

In one specific embodiment, the present invention provides a process for preparing caspofungin diacetate comprising:
(a) providing a solution of caspofungin diacetate in ethanol;
(b) removing an excess of water by evaporating a portion of the solution to achieve about 15%-25% by volume of the starting solution of step (a);
(c) adding 50% by volume of the starting solution of step (a) of anhydrous ethanol;
(d) repeating steps (b) and (c) to achieve a final water content which is less then 1% wt % and
(e) drying the caspofungin from residual solvent by evaporation to obtain powder.

Preferably the solution of caspofungin diacetate in ethanol is obtained by combining caspofungin with a mixture of ethanol and acetic acid or by combining caspofungin diacetate with ethanol.

The starting solution of a pharmaceutically acceptable salt of aza cyclohexapeptide in an organic solvent used in the above-described processes, can be prepared by a process comprising:

(a) purifying a crude peptide using preparative HPLC, and

(b) eluting the peptide with in a mixture of an organic solvent and an acid of a pharmaceutically acceptable salt.

Chemical synthesis of the crude aza cyclohexapeptides could be performed according to known methods described in the art such as WO 94/21677, WO 96/24613, U.S. Pat. No. 5,552,521, WO 97/47645, U.S. Pat. No. 5,936,062 and WO 02/083713 which are incorporated herein by reference. Example 4 below describes a preferred process for preparing the crude caspofungin.

Optionally, the crude product solution obtained at the end of the synthesis stage requires neutralizing excess of deprotecting agent or other reagent prior to purification. When the deprotecting agent is basic, neutralization may be accomplished using mineral acids or organic acids. Suitable mineral acids include, but are not limited to phosphoric acid, hydrochloric acid, sulfuric acid or nitric acid. Suitable organic acids include, but are not limited to acetic acid or trifluoroacetic acid. Preferably, the neutralization is accomplished using acetic acid.

The crude aza cyclohexapeptide may be purified by chromatography and other means of purification known in the art including, but not limited to ion exchange, crystallization, or extraction. Preferably, the purification of the peptides is performed by a preparative HPLC.

As used herein, the term crude peptide refers to the peptide obtained by synthesis process (described in example 4).

Solvents that may be used in the preparative HPLC are for example, but are not limited to acetonitrile, methanol, ethanol and propanol. Preferably, acetonitrile is used.

In one specific embodiment, the step for purifying the crude peptide by preparative HPLC includes running a mobile phase comprising organic solvents (such as acetonitrile, methanol, ethanol or propanol and aqueous buffer) through a reverse phase HPLC column packed with a stationary phase such as C-8 or C-18 silica-based resin. The mobile phase may be, for example, 0.05%-0.5% (v/v) acetic acid in water and acetonitrile. Gradient elution programs employ two solvents: an aqueous phase and an organic phase. The organic phase may be acetonitrile. Preferably, the preparative HPLC technique is reverse phase (RP)HPLC, during which the peptide is eluted by increasing the percentage of organic solvent. The organic phase during the purification cycle may vary from about 10% to about 60% as a function of time. Ultraviolet (UV) detection may occur at various wavelengths including, but not limited to, 214 nm, 220 nm or 230 nm. See, e.g., G. Grant, SYNTHETIC PEPTIDES: A USER'S GUIDE 223-227 (Oxford University Press 1992).

Optionally, prior to elution of the peptide, the peptide may be washed with a mixture of water and organic solvent to remove an excess of acid remained after purification or ion-exchange stages. Preferably the mixture contains 2-7% organic solvent, most preferably it contains 5% organic solvent.

A solvent which may be used together with water is ethanol.

Preferably, prior to the elution step of the above process, the pure peptide is maintained for a period of no longer than 1 hour in order to prevent possible degradation due to aqueous conditions. It is assumed that the degradation is caused by the presence of water particularly in the solid state and therefore the amount of water should be kept at its minimal amount at each step when solid product is obtained.

Optionally, after purifying the peptide using preparative HPLC, the counter-ion of the peptide is exchanged, on a preparative HPLC or an ion-exchange resin, to a pharmaceutically acceptable counter-ion. This option may be used when the purification step is performed without the use of a pharmaceutically acceptable counter-ion.

Suitable organic solvents for use in the elution from the column include, but are not limited to, methanol, ethanol, propanol, isopropanol, t-butyl alcohol, acetonitrile, tetrahydrofuran or mixtures of the same with water or other solvents.

One should preferably use an organic solvent which can elute the peptide from the column and also is suitable for the drying processes described above.

When preparing the solution of caspofungin in a solvent, to be used in the drying processes of the invention, the ratio of organic solvent: water is preferably about 50:50 to about 95:5. More preferably, the organic solvent is ethanol and the ratio of ethanol:water is about 80:20 to about 95:5.

Most preferably a mixture of an organic solvent and water at a ratio of about 90:10 to about 95:5 organic solvent: water is used.

The acid of a pharmaceutically acceptable salt used, is in a ratio of about 0.025% to about 0.1% of the total mixture volume. Most preferably 0.05%.

Optionally, when preparing the solution of caspofungin in a solvent, to be used in the spray drying process, the solution is concentrated prior to the feeding into the spray-dryer, for example by using a TFE (Thin film evaporator) or by any other methods of evaporation to concentrate solution prior to spray drying.

Peptides that may be produced by =the processes described above include, but are not limited to, caspofungin diacetate (SEQ. ID NO. 1).

In an exemplary embodiment demonstrating preparation of caspofungin diacetate (SEQ. ID NO. 1): (1) the crude product is produced according to any method known in the art and incorporated herein by reference; (2) the reaction solution is optionally diluted with water and neutralized to pH about 4 by addition of acetic acid; (3) the solution is loaded to the preparative HPLC and is purified using aqueous buffer containing acetic acid and acetonitrile to obtain material containing <0.1% of each process or degradation impurity; (4) the purified fractions are loaded to the preparative HPLC column followed by washing with water containing about 5% ethanol; (5) the peptide is eluted from the column by a stream of ethanol/water (95:5); (6) and the resulting solution is concentrated under reduced pressure to about half of its starting volume followed by addition of fresh anhydrous ethanol and repeating the procedure several times to obtain the minimal allowed amount of water; (7) the solvent is evaporated to dryness to obtain free flowing powder optionally followed by addition of fresh portions of ethanol and its evaporation to complete drying procedure.

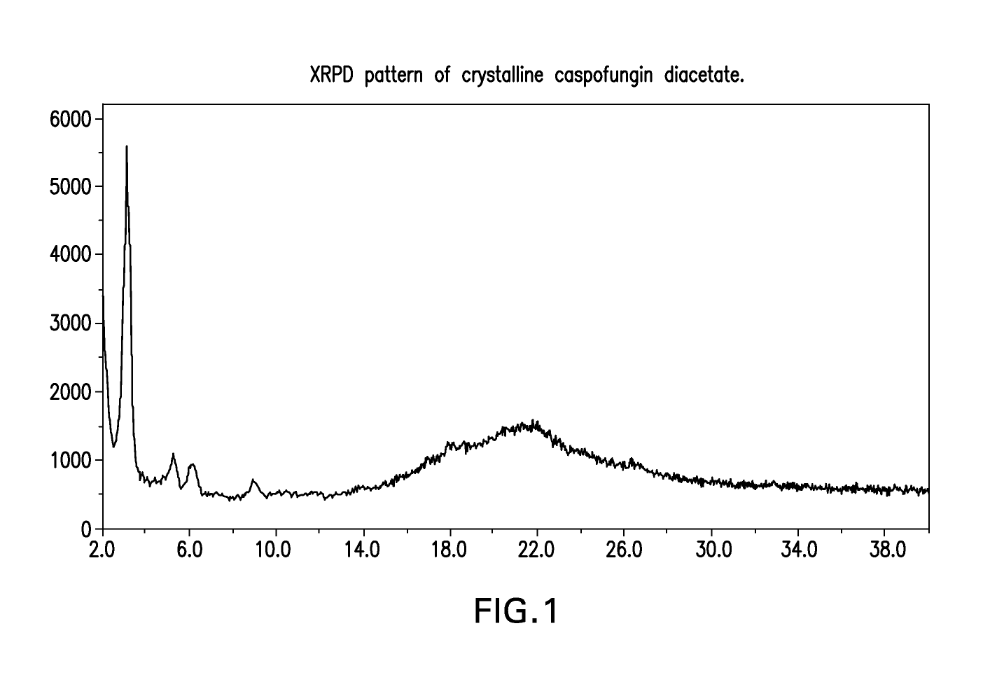

In another embodiment, the present invention encompasses a crystalline form of caspofungin diacetate, characterized by data selected from the group consisting of: a powder XRD pattern with peaks at about 3.1, 5.2, 6.1 and 9.0±0.2 degrees two-theta, a powder XRD pattern substantially as depicted in FIG. 1; and combinations thereof.

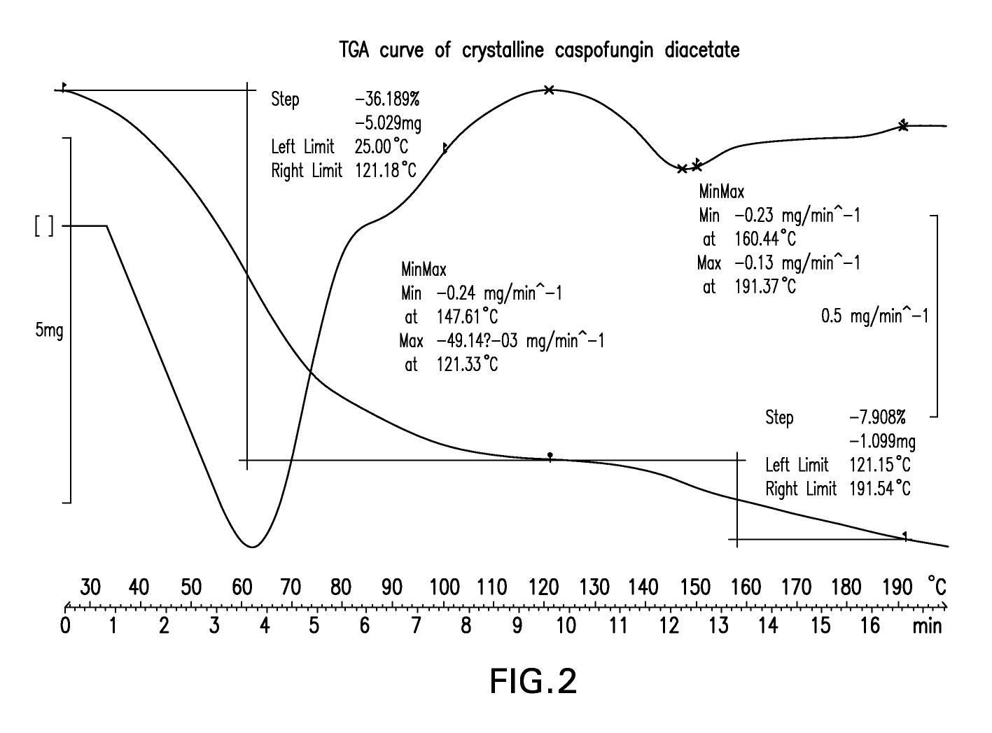

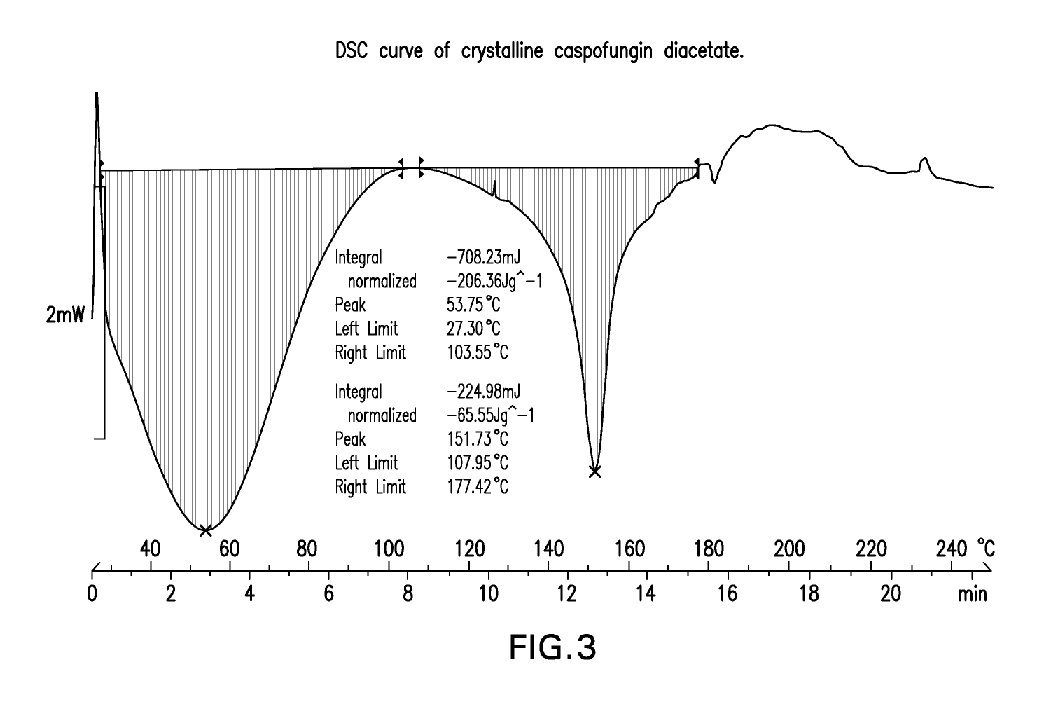

The above-described crystalline caspofungin diacetate polymorph can be further characterized by data selected from the group consisting of: a weight loss of about 36% by weight at a temperature of up to about 120° C. as measured by Thermal Gravimetric Analysis (TGA); TGA curve as depicted in FIG. 2; a water content of about 3% by weight as measured by Karl-Fisher; a Differential Scanning Calorimetry (DSC) curve with two endothermic peaks, one at about 54° C. due to desolvation, and the second at about 152° C. due to melting during decomposition; and a DSC curve as depicted in FIG. 3.

The crystalline form of caspofungin diacetate may be obtained by a process comprising:
(a) providing a solution caspofungin diacetate in an organic solvent;
(b) removing an excess of water by evaporation;
(c) adding an additional portion of anhydrous organic solvent; and
(d) repeating steps (b) and (c) one or more times to achieve a water content of more than 1% wt.
(e) drying the caspofungin from residual solvent by evaporation to obtain powder.

Preferably the solution of caspofungin diacetate in an organic solvent is obtained by combining caspofungin with a mixture of an organic solvent and acetic acid or by combining caspofungin diacetate with an organic solvent.

The present invention further encompasses 1) a pharmaceutical composition comprising any one, or combination, of the crystalline form of caspofungin diacetate and/or amorphous form described above and at least one pharmaceutically acceptable' excipient and 2) the use of any one, or combination, of the above-described crystalline form of caspofungin diacetate and/or amorphous form, in the manufacture of a pharmaceutical composition, wherein the pharmaceutical composition can be useful for the treatment of systemic fungal infections

The pharmaceutical composition can be prepared by a process comprising combining any one, or combination, of the above-described crystalline form of caspofungin diacetate and/or amorphous form with at least one pharmaceutically acceptable excipient. The crystalline form of caspofungin diacetate and/or amorphous form can be obtained by any of the processes of the present invention as described above.

Any one, or combination, of the above-described crystalline form of caspofungin diacetate and/or amorphous form of the present invention, particularly in a pharmaceutical composition and dosage form, can be used to treat systemic fungal infections caused by Candida, Aspergillus, Histoplasma, Coccidioides and Blastomyces or to treat and prevent infections caused by Pneumocystis carinii in a mammal such as a human, comprising administering a treatment effective amount of the one, or combination, of the crystalline form of caspofungin diacetate and/or amorphous form in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

Preferably, caspofungin diacetate forms or amorphous form are made by the processes of the present invention.

Having described the invention with reference to certain preferred embodiments, other embodiments will become apparent to one skilled in the art from consideration of the specification. The invention is further defined by reference to the following examples describing in detail the process for purifying peptides. It will be apparent to those skilled in the art that many modifications, both to materials and methods, may be practiced without departing from the scope of the invention.

### EXAMPLES

### Experimental Methodology (Physical)

### XRD

ARL X-ray powder diffractometer model X′TRA-030, equipped with Cu irradiation source λ=1.54178, Peltier detector, round standard aluminum sample holder with round zero background quartz plate was used. Scanning parameters: Range: 2-40 deg. 2θ, continuous Scan, Rate: 3 deg./min. The accuracy of peak positions is defined as +/−0.2 degrees due to experimental differences like instrumentations, sample preparations etc.

### TGA

Mettler 3M with Mettler TG 50 thermobalance

Heating rate: 10° C./min., In N₂ stream: flow rate=40 ml/min

Scan range: 25-200° C., Sample weight 7-15 mg

### DSC

Mettler Toledo DSC 821^{e} calorimeter

Heating rate: 10° C./min., In N₂ stream: flow rate=40 ml/min

Number of holes of the crucible: 3

Scan range: 25-250° C., Sample weight: 3-5 mg

### KF

Mettler Toledo DL 38 Karl Fisher Titrator

Sample weight: 50-150 mg

### Spray Drying

Spray drying was performed using a Buchi mini spray dryer B-290 using a standard nozzle 0.7 mm in diameter with a nozzle cap of 1.4 or 1.5 mm.

### Preparative HPLC for Purifying Caspofungin

| | |
|---|---|
| Column: | Silica C-18, 6 inch |
| Eluent: | A - 1% acetic acid in water |
| | B - acetonitrile |
| Flow rate: | 1 l/min |
| Column temperature: | RT (room temperature) |
| Run time: | ~60 min |
| Wavelength: | 230 nm |

### Example 1

### Preparation of Caspofungin Diacetate (SEQ. ID NO. 1) Via Lyophilization Drying

### Reference Example

Crude caspofungin solution obtained after last reaction stage, exchange of protected thiol group by ethylenediamine group, (about 30 g of solid) was diluted with water and neutralized to pH about 4 by addition of acetic acid. The solution was loaded to preparative HPLC column packed with C-18 resin and purified using acetonitrile/water buffer containing 0.5% acetic acid. Fractions containing >99.0% pure product and each impurity <0.1% were combined and loaded to lyopilizer. The resulted powder was 98.5% pure and it contained degradation impurities at level <0.5% each.

### Example 2

### Preparation of Caspofungin Diacetate (SEQ. ID NO. 1) via Evaporation Drying

Crude caspofungin solution obtained after last reaction stage, exchange of protected thiol group by ethylenediamine group, (about 30 g of solid) was diluted with water and adjusted to pH about 4 by addition of acetic acid. The solution was loaded to preparative HPLC column packed with C-18 resin and purified using acetonitrile/water buffer containing 0.5% acetic acid. Fractions containing >99.0% pure product and each impurity <0.1% were combined and loaded to the preparative column previously washed with ethanol and equilibrated with 95% water/ethanol solution. The column after loading was washed with 95% water/ethanol solution. The material was eluted from the column by a stream of 95% ethanol/water containing 0.05% acetic acid. The caspofungin solution was evaporated to about half the starting volume and diluted to the starting volume with fresh anhydrous ethanol. This step was repeated four times until the water content was 1%≧wt % and then the solvent was evaporated to dryness. Moderate heating (up to 40 C) was also used in this step. The solid was dissolved again by addition of the fresh anhydrous Ethanol and evaporated to dryness to obtain free-flowing powder of amorphous caspofungin diacetate. The purity of the product was >99.0% (HPLC) and each impurity was in the range of <0.1% to <0.25%.

### Example 3

### Preparation of Caspofungin Diacetate (SEQ. ID NO. 1) via Spray Drying

Crude caspofungin solution obtained after last reaction stage, exchange of protected thiol group by ethylenediamine group, (about 114 g of solid) was diluted with water and adjusted to pH about 4 by addition of acetic acid. The solution was loaded to preparative HPLC column packed with C-18 resin and purified using water/acetonitrile buffer containing 0.5% acetic acid. Fractions containing >99.0% pure product and each impurity <0.1% were combined and loaded to a preparative column previously washed with ethanol and equilibrated with 95% water/ethanol solution The column after loading was washed with 95% water/ethanol solution. The material was eluted from the column by a stream of 95% ethanol/water containing 0.05% acetic acid.

8.5 liter of HPLC preparative solution was fed to Spray-dryer at rate of 750 ml/hr. Then the product from collection vessel was discharged every 30 minutes and kept under cold condition of −70° C. The nitrogen gas was at an inlet temperature of 80° C. The evaporated solvent and nitrogen left the spray dryer chamber at a temperature of 35° C. Product collected weight was 104 gr. purification yield: 60%, drying yield: 91%, total yield: 54%.

### Example 4

### Preparation of Crude Caspofungin

### Reference Example

One sample of pneumocandin Bo purified by silica gel column chromatography was transformed to caspofungin according to the following steps:

Preparation of 4-methoxyphenylthio-pneumocandin Bo

Pneumocandin Bo (25.2 g) (assay: 89.3%; HPLC) was suspended in acetonitrile (630 ml) in a jacketed reactor fitted with thermometer, nitrogen inlet and mechanical stirrer. The mixture was cooled to −15 C.° by means of a thermostat, and 4-methoxythiophenol (5.88 g) was added in one portion. Trifluoroacetic acid (117.9 g) was added dropwise in about 20 min keeping the temperature between −10÷-15 C.°.

The mixture was stirred at −15 C.° for 22 h and quenched by addition of water (1260 ml) at a temperature bellow 0 C.° in about 60 min. The mixture was stirred at about 0 C.° for 1 h then the precipitated solid was collected, washed twice with acetonitrile—water (1:3 v/v) (140 and 140 ml) and twice with acetonitrile (105 and 70 ml) to afford the product 23.97 g (85.2%) after drying in vacuum at less than 40 C.° for 24 h in the HPLC purity of 78.8 A % and assay of 72.2%.

### Preparation of 4-methoxyphenylthio-pneumocandin Bo amine

4-Methoxyphenylthio-pneumocandin Bo (14.0 g) was suspended in tetrahydrofuran (500 ml) then phenylboronic acid (2.31 g) was added, and the mixture was stirred at less than 40 C.° until obtaining a solution (4 h). Molecular sieve of 3 Å (50 g) was then added to the mixture and was allowed to stand at room temperature for about 16 h to decrease the water content (LT 150 ppm).

The molecular sieve was removed, washed with THF (50 ml) and the filtrate was charged to a jacketed reactor fitted with nitrogen inlet, thermometer and a thermostat.

The solution was cooled to −5 C.° and borane-dimethylsulfide complex (3.86 g/90% pure) was added in about 15 min at 0÷−5 C.° resulting in a dense gelatinous mixture in 30 min after addition which was stirred at about −5 C.° for 10 h.

The reaction mixture was cooled to −15 C.°, and quenched by addition of 2N aqueous hydrochloric acid solution (8 ml) at (−10)-(−15) C.° in about 15 min resulting in a clear solution.

The quenched mixture was stored in a freezer at about −15 C.° overnight, then was diluted with water (2200 ml).

The diluted solution was filtered through a sintered glass filter and charged onto a 295 g reverse phase (LiChroprep RP-18, Merck) medium pressure column (36×460 mm) with the speed of about 18 ml/min. The column was washed with acetonitrile—water (20:80 v/v; 1800 ml; 18 ml/min) and the product was eluted with acetonitrile—water (40:60 v/v; about 14 ml/min). Fractions of 200 ml each were collected by means of a fraction collector and analyzed by TLC, than the fractions showing the presence of the product, by HPLC. The rich cuts (>88 A %) were combined, diluted with water and charged to a 125 g of a reverse phase column (LiChroprep RP-18, Merck).

The product was eluted with methanol by means of gravitation, collecting 5×120 ml fraction which were analyzed by HPLC. The suitable fractions were combined and concentrated on a rotary evaporator at a temperature of less than 30 C.° and the product was precipitated by addition of acetonitrile.

The mixture was cooled to 2-8 C.°, the solid was collected, washed with acetonitrile (20 ml) and dried in a vacuum oven at room temperature for 24 h to yield 4.82 g (35.2%) of the product in a HPLC purity of 96.8 A % and assay of 91.9%.

### Preparation of Caspofungin Diacetate

4-Methoxyphenylthio-pneumocandin Bo amine (4.34 g) was added to ethylenediamine (18.5 ml) under nitrogen while stirring and cooling at 15-25 C.°. The mixture was stirred at room temperature for 6 h then it was diluted with methanol (24 ml) while cooling with ice-water at 15-25 C.°. The mixture of water (90 ml) and acetic acid (24 ml) was added under the same condition, and finally, the pH of the mixture was adjusted to 6-7 by addition of acetic acid (8 ml).

What is claimed is:

1. A process for preparing aza cyclohexapeptide salts comprising spray drying a solution of a pharmaceutically acceptable salt of aza cyclohexapeptide in an organic solvent.

2. The process of claim 1 wherein the solution of a pharmaceutically acceptable salt of aza cyclohexapeptide in an organic solvent is obtained by combining an aza cyclohexapeptide with a mixture of an organic solvent and an acid of a pharmaceutically acceptable salt or by combining a pharmaceutically acceptable salt of aza cyclohexapeptide with an organic solvent.

3. The process of claim 1, wherein the solution of a pharmaceutically acceptable salt of aza cyclohexapeptide in an organic solvent is prepared by a process comprising:
(a) purifying a crude peptide using preparative HPLC, and
(b) eluting the peptide in a mixture of organic solvent and an acid of a pharmaceutically acceptable salt.

4. The process of claim 1, wherein the organic solvent is selected from the group consisting of: ethanol, acetonitrile, methanol, propanol, isopropanol, t-butyl alcohol, tetrahydrofuran and mixtures of organic solvent and water.

5. The process of claim 4, wherein the organic solvent is ethanol.

6. The process of claim 4, wherein the organic solvent:water ratio is about 50:50 to about 95:5.

7. The process of claim 4, wherein the organic solvent:water ratio is about 80:20 to about 95:5 organic solvent:water.

8. The process of claim 4, wherein the organic solvent:water ratio is about 90:10 to about 95:5 organic solvent:water.

9. The process of claim 3, wherein the amount of the acid of a pharmaceutically acceptable salt in the mixture is about 0.025% to about 0.1% of the total mixture volume of the eluent.

10. The process of claim 9, wherein the amount of the acid of a pharmaceutically acceptable salt in the mixture is about 0.05% of the total mixture volume.

11. The process of claim 1 wherein the solution of a pharmaceutically acceptable salt of aza cyclohexapeptide in an organic solvent is spray-dried at an inlet temperature of from about 50° C. to about 200° C.

12. The process of claim 11 wherein the solution of a pharmaceutically acceptable salt of aza cyclohexapeptide in an organic solvent is spray-dried at an inlet temperature of from about 50° C. to about 150° C.

13. The process of claim 12 wherein the solution of a pharmaceutically acceptable salt of aza cyclohexapeptide in an organic solvent is spray-dried at an inlet temperature of about 80° C.

14. The process of claim 1, wherein the solution of a pharmaceutically acceptable salt of aza cyclohexapeptide in an organic solvent is spray-dried at an outlet temperature of from about 10° C. to about 65° C.

15. The process of claim 14, wherein the solution of a pharmaceutically acceptable salt of aza cyclohexapeptide in an organic solvent is spray-dried at an outlet temperature of from about 20° C. to about 55° C.

16. The process of claim 15, wherein the solution of a pharmaceutically acceptable salt of aza cyclohexapeptide in an organic solvent is spray-dried at an outlet temperature of from about 30° C. to about 45° C.

17. The process of claim 1, wherein the salt is selected from the group consisting of: citrate, acetate, palmitate, trifluoroacetate, hydrochloride, maleate, tartrate, succinate, oxalate, malate or glutamate.

18. The process of claim 1, wherein the aza cyclohexapeptide is selected from the group consisting of: caspofungin, anidulafungin and micafungin.

19. The process of claim 1, wherein the aza cyclohexapeptide salt is caspofungin diacetate.

20. The process of claim 19, wherein the obtained caspofungin diacetate has a purity of at least about 99.0% as measured by area HPLC.

21. The process of claim 20 wherein the obtained caspofungin diacetate has a purity of at least about 99.5% as measured by area HPLC.

22. The process of claim 20, wherein the obtained caspofungin diacetate has less than about 0.25% as measured by area HPLC of each degradation impurity including CPF-Dimer 1 (at RRT 2.08) and CPF-dimer 2 (at RRT 2.26).

23. The process of claim 22 wherein the obtained caspofungin diacetate has less than 0.1% of each degradation impurity, and degradation impurity including CPF-Dimer 1 (at RRT 2.08) and CPF-dimer 2 (at RRT 2.26).

24. The process of claim 19 wherein the obtained caspofungin diacetate has less than 5% of crystalline caspofungin diacetate, as measured by XRD.

25. The process of claim 24 wherein the obtained caspofungin diacetate has less than 1% of crystalline caspofungin diacetate, as measured by XRD.

26. The process of claim 25 wherein the obtained caspofungin diacetate has less than 0.5% of crystalline caspofungin diacetate, as measured by XRD.

27. The process of claim 24, wherein the obtained caspofungin diacetate has less than 1% of crystalline form of caspofungin diacetate, characterized by data selected from the group consisting of: a powder XRD pattern with peaks at about 3.1, 5.2, 6.1 and 9.0±0.2 degrees two-theta, a powder XRD pattern substantially as depicted in FIG. 1; and combinations thereof, as measured by XRD.

28. The process of claim 19, wherein the obtained caspofungin diacetate is amorphous.

29. The process of claim 1, wherein there is an excess amount of acid present in the solution.

30. A process for preparing caspofungin diacetate comprising spray drying a solution of caspofungin diacetate in ethanol.

31. The process of claim 30, wherein the solution of caspofungin diacetate in ethanol is obtained by combining caspofungin with a mixture of ethanol and acetic acid or by combining a caspofungin diacetate with ethanol.

32. A process for preparing aza cyclohexapeptide salts comprising the steps of:
(a) providing a solution of a pharmaceutically acceptable salt of aza cyclohexapeptide in an organic solvent;
(b) removing an excess of water by evaporating a portion of the solution;
(c) adding anhydrous organic solvent; and
(d) repeating steps (b) and (c) one or more times.

33. The process of claim 32, wherein there is an excess amount of acid present in the solution.

34. The process of claim 32 wherein the solution of a pharmaceutically acceptable salt of aza cyclohexapeptide in an organic solvent is obtained by combining an aza cyclohexapeptide with a mixture of an organic solvent and an acid of a pharmaceutically acceptable salt or by combining a pharmaceutically acceptable salt of aza cyclohexapeptide with an organic solvent.

35. The process of claims 32, wherein the solution of a pharmaceutically acceptable salt of aza cyclohexapeptide in an organic solvent is prepared by a process comprising:
(a) purifying a crude peptide using preparative HPLC, and
(b) eluting the peptide in a mixture of organic solvent and an acid of a pharmaceutically acceptable salt.

36. The process of claim 32, wherein the organic solvent is selected from the group consisting of: ethanol, water, acetonitrile, methanol, propanol, isopropanol, t-butyl alcohol, tetrahydrofuran and mixtures of organic solvent and water.

37. The process of claim 36, wherein the organic solvent is ethanol.

38. The process of claim 36, wherein the organic solvent:water ratio is about 50:50 to about 95:5.

39. The process of claim 36, wherein the organic solvent:water ratio is about 80:20 to about 95:5 organic solvent:water.

40. The process of claim 36, wherein the organic solvent:water ratio is about 90:10 to about 95:5 organic solvent:water.

41. The process of claims 33, wherein the amount of the acid of a pharmaceutically acceptable salt in the mixture is 0.025% to about 0.1% of the total mixture volume.

42. The process of claim 33, wherein the amount of the acid of a pharmaceutically acceptable salt in the mixture is 0.05% of the total mixture volume.

43. The process of claim 32 wherein the repeating is stopped when the final water content is less then 1% by, wt %.

44. The process of claim 32 wherein the final water content is measured using KF (Karl Fischer).

45. The process of claim 32, wherein in step (b) the solution is evaporated to achieve about 15% to about 25% by volume of the starting solution.

46. The process of claim 45, wherein in step (b) the solution is evaporated to achieve about 20% by volume of the starting solution.

47. The process of claim 32, wherein a total of about 1 volume to about 3 volumes of the starting solution of the anhydrous organic solvent is added to the solution.

48. The process of claim 47, wherein a total of about 2 volumes of the starting solution of the anhydrous organic solvent is added to the solution.

49. The process of claim 32, wherein the salt is selected from the group consisting of: citrate, acetate, palmitate, trifluoroacetate, hydrochloride, maleate, tartrate, succinate, oxalate, malate or glutamate.

50. The process of claims 49 wherein the aza cyclohexapeptide salt is caspofungin diacetate.

51. The process of claim 50 wherein the obtained caspofungin diacetate has a purity of at least about 99.0% as measured by HPLC area.

52. The process of claim 51 wherein the obtained caspofungin diacetate has a purity of at least about 99.5% as measured by HPLC area.

53. The process of claim 51, wherein the obtained caspofungin diacetate has less than 0.25% area by HPLC of each process and degradation impurity including CPF-Dimer 1 and CPF-dimer 2.

54. The process of claim 53 wherein the obtained caspofungin diacetate has less than 0.1% area by HPLC of each degradation impurity and degradation impurity including CPF-Dimer 1 (at RRT 2.08) and CPF-dimer 2 (at RRT 2.26).

55. The process of claim 50 wherein the obtained caspofungin diacetate has less than 5% of crystalline caspofungin diacetate, as measured by XRD.

56. The process of claim 55 wherein the obtained caspofungin diacetate has less than 1% of crystalline caspofungin diacetate, as measured by XRD.

57. The process of claim 56 wherein the obtained caspofungin diacetate has less than 0.5% of crystalline caspofungin diacetate, as measured by XRD.

58. The process of claim 51, wherein the obtained caspofungin diacetate has less than 1% of crystalline form of caspofungin diacetate, characterized by data selected from the group consisting of: a powder XRD pattern with peaks at about 3.1, 5.2, 6.1 and 9.0±0.2 degrees two-theta, a powder XRD pattern substantially as depicted in FIG. 1; and combinations thereof, as measured by XRD.

59. The process of claim 50, wherein the obtained caspofungin diacetate is amorphous.

60. A process for preparing caspofungin diacetate comprising:
(a) providing a solution of caspofungin diacetate in ethanol;
(b) removing an excess of water by evaporation to achieve about 20% by volume of the starting solution;
(c) adding 50% by volume of the starting solution of anhydrous ethanol;
(d) repeating steps (b) and (c) four times and
(e) drying the caspofungin from residual solvent by evaporation to obtain powder.

61. The process of claim 60, wherein there is an excess amount of acid present in the solution.

62. The process of claim 60 wherein the solution of caspofungin diacetate in ethanol is obtained by combining caspofungin with a mixture of ethanol and acetic acid or by combining caspofungin diacetate with ethanol.

63. A crystalline form of caspofungin diacetate, characterized by data selected from the group consisting of: a powder XRD pattern with peaks at about 3.1, 5.2, 6.1 and 9.0±0.2 degrees two-theta, a powder XRD pattern substantially as depicted in FIG. 1; and combinations thereof.

64. A pharmaceutical composition comprising any one, or combination, of the crystalline form of caspofungin diacetate and/or amorphous form as defined in any of the preceding claims, and at least one pharmaceutically acceptable excipient.

65. The pharmaceutical composition of claim 64, wherein the pharmaceutical composition is prepared by a process comprising combining the crystalline form of caspofungin diacetate and/or amorphous form as defined in any of the preceding claims, with at least one pharmaceutically acceptable excipient.

66. A method of treatment comprising administering to a mammal in need thereof a pharmaceutical composition comprising any one, or combination, of the crystalline form of caspofungin diacetate and/or amorphous form as defined in any of the preceding claims, for the treatment of systemic fungal infections caused by Candida, Aspergillus, Histoplasma, Coccidioides and Blastomyces.

67. A method of treatment comprising administering to a mammal in need thereof a pharmaceutical composition comprising any one, or combination, of the crystalline form of caspofungin diacetate and/or amorphous form as defined in any of the preceding claims, for the treatment and prevention of infections caused by Pneumocystis carinii.
